(19) United States
(12) Patent Application Publication
(10) Pub. No.: US 2013/0184697 A1
(43) Pub. Date: Jul. 18, 2013

(54) SYSTEM AND METHOD FOR NON-INVASIVE TREATMENT OF CARDIAC ARRHYTHMIAS

(71) Applicant: GENERAL ELECTRIC COMPANY, Schenectady, NY (US)

(72) Inventors: Xiaodong Han, Shanghai (CN); Yao Chen, Shanghai (CN); Weihua Gao, Shanghai (CN); Joel Qiuzhen Xue, Germantown, WI (US); Yu Zhang, ShangHai (CN); Peng Zhu, ShangHai (CN)

(73) Assignee: GENERAL ELECTRIC COMPANY, Schenectady, NY (US)

(21) Appl. No.: 13/738,451

(22) Filed: Jan. 10, 2013

(30) Foreign Application Priority Data
Jan. 12, 2012 (CN) 201210008379.1

(51) Int. Cl. A61B 19/00; A61B 18/18

(52) U.S. Cl. CPC A61B 19/50; A61B 18/18
USPC 606/33; 606/32

(57) ABSTRACT

A method for non-invasive treatment of cardiac arrhythmias is provided. The method includes acquiring body surface electrical signals at locations on a body surface of a living being from electrodes placed on locations of the body surface, reconstructing three-dimensional heart and torso anatomical models of the living being from an imaging scan, and calculating an electrical activity a throughout three-dimensional volume of the heart by electrocardiogram inverse problem solving based at least in part on the acquired body surface electrical signals and the reconstructed three-dimensional heart and torso anatomical models. The method also includes identifying at least one location of at least one site of origin of a cardiac arrhythmia according to the calculated electrical activity within the heart, and delivering focused energy to the identified at least one location of the at least one site of origin of the cardiac arrhythmia.

### BACKGROUND OF THE INVENTION

Embodiments of the present invention relate generally to the treatment of cardiac arrhythmias, and, in particular, a non-invasive treatment of cardiac arrhythmias.

Conventional treatment of cardiac arrhythmias includes drugs used for rate control, maintenance of sinus rhythm, and stroke prevention. Due to the poor efficacy and potential side effects associated with drug treatment, catheter based ablation has been developed for treatment of the cardiac arrhythmias Catheter based ablation treatment involves inserting catheters into a blood vessel through a site in the groin or neck and threading them through the vein until reaching the heart. The electrodes on the tip of the catheters can be used to conduct an electrophysiology (EP) study to pinpoint the location of the arrhythmia sites. Then, radio frequency (RF) or other modalities energy can be transmitted to destroy the abnormal cardiac tissue. However, the catheter based ablation for cardiac arrhythmia treatment is an invasive procedure, which may cause patient discomfort and pain, infection, and long hospital stays.

### BRIEF DESCRIPTION OF THE INVENTION

In accordance with an embodiment, systems and methods are provided for non-invasive treatment of cardiac arrhythmias. One exemplary method provided includes acquiring body surface electrical signals at locations on a body surface of a living being from electrodes placed on the locations of the body surface, reconstructing three-dimensional heart and torso anatomical models of the living being from an imaging scan, and calculating an electrical activity throughout a three-dimensional volume of the heart by electrocardiogram inverse problem solving based at least in part on the acquired body surface electrical signals and the reconstructed three-dimensional heart and torso anatomical models. The method also includes identifying at least one location of at least one site of origin of a cardiac arrhythmia according to the calculated electrical activity within the heart, and delivering focused energy to the identified at least one location of the at least one site of origin of the cardiac arrhythmia.

According to an embodiment, a system for non-invasive treatment of cardiac arrhythmias is provided. The system comprises an imaging device, a processor, and a focused energy delivering device. The imaging device is configured to perform an image scan for reconstruction of three-dimensional heart and torso models of a living being. The processor comprises a body surface electrical potential acquiring module configured to acquire body surface electrical signals at locations on a body surface of a living being from electrodes placed on the locations of the body surface, an electrical activity calculation module configured to calculate an electrical activity throughout a three-dimensional volume of the heart by electrocardiogram inverse problem solving based at least in part on the acquired body surface electrical signals and the three-dimensional heart and torso models, a cardiac arrhythmia analyzing module configured to identify at least one location of at least one site of origin of a cardiac arrhythmia based at least in part on the calculated electrical activity throughout the three-dimensional volume of the heart The focused energy delivering device is configured to deliver focused energy to the at least one location of the at least one site of origin of the cardiac arrhythmia.

According to an embodiment, a non-transitory computer-readable medium comprising instructions stored thereon, which when executed by a processor of a system for non-invasive treatment of cardiac arrhythmia perform a method, is provided. The method comprises acquiring body surface electrical signals at locations on a body surface of a living being from electrodes placed on the locations of the body surface, and reconstructing three-dimensional heart and torso anatomical models of a heart and a torso of the living being from an imaging scan. The method further comprises calculating an electrical activity throughout a three-dimensional volume of the heart by electrocardiogram inverse problem solving based at least in part on the acquired body surface electrical signals and the three-dimensional heart and torso models, identifying at least one location of at least one site of origin of the cardiac arrhythmia based at least in part on the calculated electrical activity throughout the three-dimensional volume of the heart, and delivering focused energy to the at least one location of the at least one site of origin of the cardiac arrhythmia.

### BRIEF DESCRIPTION OF THE DRAWINGS

These and other features and aspects of embodiments of the present disclosure will become better understood when the following detailed description is read with reference to the accompanying drawings in which like characters represent like parts throughout the drawings, wherein:

FIG. 1 is a block diagram of an overall system for non-invasive treatment of cardiac arrhythmias in accordance with an exemplary embodiment of the present disclosure;

FIG. 2 is a block diagram illustrating a non-invasive mapping sub-system of the overall system shown in FIG. 1 for mapping of the cardiac arrhythmia sites in accordance with an exemplary embodiment of the present disclosure;

FIG. 3 is a block diagram illustrating a non-invasive treatment sub-system of the overall system shown in FIG. 1 for non-invasive treatment of the cardiac arrhythmia in accordance with an exemplary embodiment of the present disclosure;

FIG. 4 is a block diagram illustrating a non-invasive assessment system for assessing cardiac arrhythmia in accordance with an exemplary embodiment of the present disclosure;

FIG. 5 is a flowchart illustrating at least some actions of a high level method for non-invasive treatment of cardiac arrhythmias in accordance with an exemplary embodiment of the present disclosure;

FIG. 6 is a flowchart illustrating at least some actions of a method for non-invasive treatment of cardiac arrhythmias in accordance with another exemplary embodiment of the present disclosure; and

FIG. 7 is a flowchart illustrating at least some actions of a method for non-invasive assessment of cardiac arrhythmias in accordance with another exemplary embodiment of the present disclosure.

### DETAILED DESCRIPTION OF THE INVENTION

Exemplary embodiments disclosed herein are generally directed to systems and methods for non-invasive treatment of cardiac arrhythmias within a heart. As used herein, “non-invasive treatment” means that all of the procedures involved for mapping, ablation, and assessment or evaluation of the cardiac arrhythmias within the heart are conducted in a manner without damaging intervening body structures or tissues. As used herein, “cardiac arrhythmias” refers to any of a large and heterogeneous group of conditions in which there is abnormal electrical activity in the heart. The cardiac arrhythmias may include, but are not limited to, supraventricular and ventricular arrhythmia, ventricular tachycardia, atrial flutter, and ventricular fibrillation.

More particularly, to non-invasively map the origin of a site of a cardiac arrhythmia within the heart, the electrical activity within the heart is calculated. As used herein, “electrical activity within the heart” refers to one or more of cardiac electrical source distribution, cardiac electrical activation sequence, excitation pattern, and electrical potentials throughout the three-dimensional volume of the heart. The three-dimensional electrical activity within the heart can be calculated by solving an electrocardiogram inverse problem based at least in part on body surface electrical signals (for example, electrical potentials) acquired at the body surface and three-dimensional anatomical torso and heart models reconstructed by performing an image scan (for example, magnetic resonance imaging scan, computed tomography scan, and ultrasound scan). After the electrical activity within the heart is calculated, the site of origin of arrhythmia can be localized, determined or identified, and focused energy (for example, high-intensity focused ultrasound energy, radio frequency energy, microwave energy, and laser energy) can be delivered to the site of origin of the arrhythmia through a focused energy delivering device to form one or more lesions at the target site. More specifically, the focused energy delivery is guided using an imaging device such as an MRI device.

In some embodiments, during the process of focused energy delivery to the target site, various parameters including the temperature, RF current field distribution, tissue elasticity, and lesion level at the target site or areas surrounding the target site can be non-invasively monitored in real-time by a variety of parameter monitoring devices (for example, MRI device for temperature, RF current field distribution, and lesion level monitoring, ultrasound device for tissue elasticity monitoring). This allows for appropriate parameter adjustments to be made to deliver optimized focused energy to the site of the origin of the arrhythmia to achieve better treatment effect of the arrhythmia ablation while minimizing tissue lesions. In some embodiments, prior to the arrhythmia localization procedure, during the arrhythmia ablation procedure, or following the arrhythmia ablation procedure, a stimulation device (for example, MRI device) can be used to generate and emit a focused magnetic field at one or more sites of the heart to actively stimulate the electrical activity within the heart. With such a stimulation mechanism, the abnormal site of the arrhythmia can be more effectively identified, and assessment can be made to confirm whether the arrhythmia ablation is effectively performed.

Embodiments of the present disclosure will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Unless defined otherwise, technical and scientific terms used herein have the same meaning as is commonly understood by one of ordinary skill in the art to which this disclosure belongs. The terms “first”, “second”, and the like, as used herein do not denote any order, quantity, or importance, but rather are used to distinguish one element from another. Also, the terms “a” and “an” do not denote a limitation of quantity, but rather denote the presence of at least one of the referenced items. The term “or” is meant to be inclusive and mean either or all of the listed items. The use of “including,” “comprising,” or “having,” and variations thereof herein are meant to encompass the items listed thereafter and equivalents thereof as well as additional items. The terms “connected” and “coupled” are not restricted to physical or mechanical connections or couplings, and can include electrical connections or couplings, whether direct or indirect. Furthermore, the terms “circuit”, “circuitry”, and “controller” may include either a single component or a plurality of components, which are either active and/or passive and are connected or otherwise coupled together (for example, as one or more integrated circuit chips) to provide the described function.

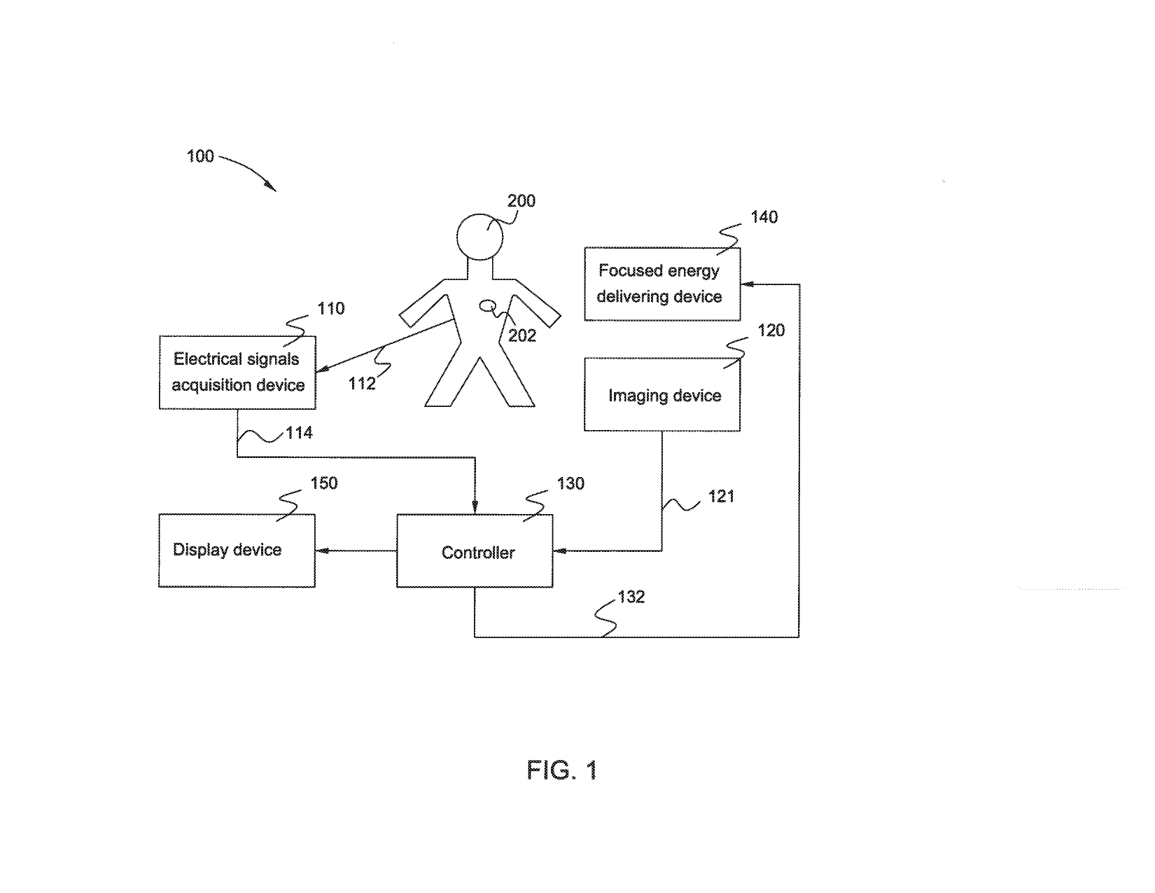

FIG. 1 is an overall block diagram illustrating a cardiac arrhythmia treatment system 100 for non-invasive treatment of cardiac arrhythmias of a living being in accordance with an exemplary embodiment of the present disclosure. As illustrated in FIG. 1, the living being is shown as a human body 200 having a heart 202. It is readily understood that the underlying principles disclosed herein could be used for non-invasive treatment of any other bio-systems (for example, animals having a heart) or organs (for example, human brain) with abnormal electrical activities.

In the illustrated embodiment, the cardiac arrhythmia treatment system 100 generally includes an electrical signal acquisition device 110, an imaging device 120 such as a magnetic resonance imaging (MRI) device, a computed tomography (CT) device, or an ultrasound device, a controller 130, a focused energy delivering device 140 such as a high-intensity focused energy (HIFU) device, a radio frequency device, a micro-wave device, or a laser device, and a display device 150. The electrical signal acquisition device 110 is configured to acquire electrical signals 112 at the body surface of the human body 200. In an embodiment, the electrical signals 112 may include electrical potentials originating from electrical activity generated within the heart 202 and conducted to the body surface through the torso and other body structures.

The imaging device 120 is configured to perform an image scan to acquire image data of the human body 200. The imaging device 120 is further configured to reconstruct three-dimensional torso and heart models of the human body 200 by processing the image data. The imaging device 120 provides data signals 121 representing the reconstructed three-dimensional torso and heart models to the controller 130. In some embodiments, for guiding the focused energy delivery during the arrhythmia ablation procedure which will be described hereinafter, the imaging device 120 may be further configured to monitor a temperature of the target site in real-time, such that over-treatment or under-treatment of site of origin of the arrhythmia can be reduced or avoided and effective treatment of the arrhythmia can be achieved.

The controller 130 may include at least one processor and at least one memory device associated with the at least one processor. The memory devices can be configured with software instructions stored therein, which when executed by the at least one processor, perform a variety of functions. For example, the controller 130 can be configured to perform a first function of calculating electrical activities within the heart by solving an electrocardiogram inverse problem based at least in part on the acquired electrical signals 114 and data signals 121 representing the reconstructed three-dimensional anatomical models of the torso and heart. The controller 130 is further configured to perform a second function of determining or identifying the site of origin of cardiac arrhythmias based on the calculated electrical activities. With the identified site of origin of the arrhythmia, the controller 130 can send control signals 132 to direct the focused energy device 140 to generate and deliver focused energy to the at least one arrhythmia site. The focused energy can create a temperature increase at the arrhythmia site, resulting in tissue ablation of the arrhythmia site by forming at least one lesion at the arrhythmia site, such that abnormal electrical activities at the arrhythmia site can be blocked or terminated.

The display device 150 is configured to display the calculated electrical activities together with the images of the heart and torso. In some embodiments, to avoid some critical structures (for example, blood vessels), the controller 130 may be configured to plan an optimal pathway for delivering the focused energy. The display device 150 is further configured to display the images of the arrhythmia site that is being treated with focused energy. From the displayed images of the arrhythmia site, medical personnel can determine whether an optimized dose of focused energy has been delivered to the arrhythmia site.

Furthermore, in some embodiments, to provide better treatment effect of the arrhythmia, a variety of parameters at the target sites receiving focused energy can be monitored in real-time. In an embodiment, the imaging device 120 as described above (for example, MRI device) may be configured to perform a special image scan to obtain temperature information of the site receiving focused energy. From the monitored temperature information, it can be determined whether the arrhythmia site is under-treated or over-treated, and then real-time parameter adjustments can be made to the focused energy delivering device 140 to deliver optimized focused energy to the arrhythmia site. For example, when the arrhythmia site is determined to be under-treated, the intensity of the focused energy generated from the focused energy delivering device can be increased, or the time duration for the focused energy delivery can be extended. In other embodiments, other parameters can be monitored for facilitating real-time parameter adjustments of the focused energy delivering device 140 for optimized focused energy delivery. For example, when the focused energy delivering device 140 is a RF energy device for generating and emitting RF energy for ablation of the one or more sites that are identified to be the origin of cardiac arrhythmia, the RF current field distribution at the target site can be non-invasively monitored in real-time by, for example, an MRI device. Still in another embodiment, the tissue elasticity at the target site can be non-invasively monitored in real-time by, for example, an ultrasound device. Further in another embodiment, the lesion level at the target site can be non-invasively monitored by, for example, a MRI device.

In some embodiments, following the arrhythmia ablation treatment procedure, the cardiac arrhythmia treatment system 100 could also be configured with some mechanisms to evaluate or assess the treatment effect of the arrhythmia sites that have been delivered with focused energy. In an embodiment, the imaging device 120 is configured to electrically stimulate the heart by emitting a focused magnetic field to the heart. Then, by solving an electrocardiogram inverse problem, the stimulation electrical activity within the heart can be calculated to determine whether the arrhythmia ablation is effectively performed.

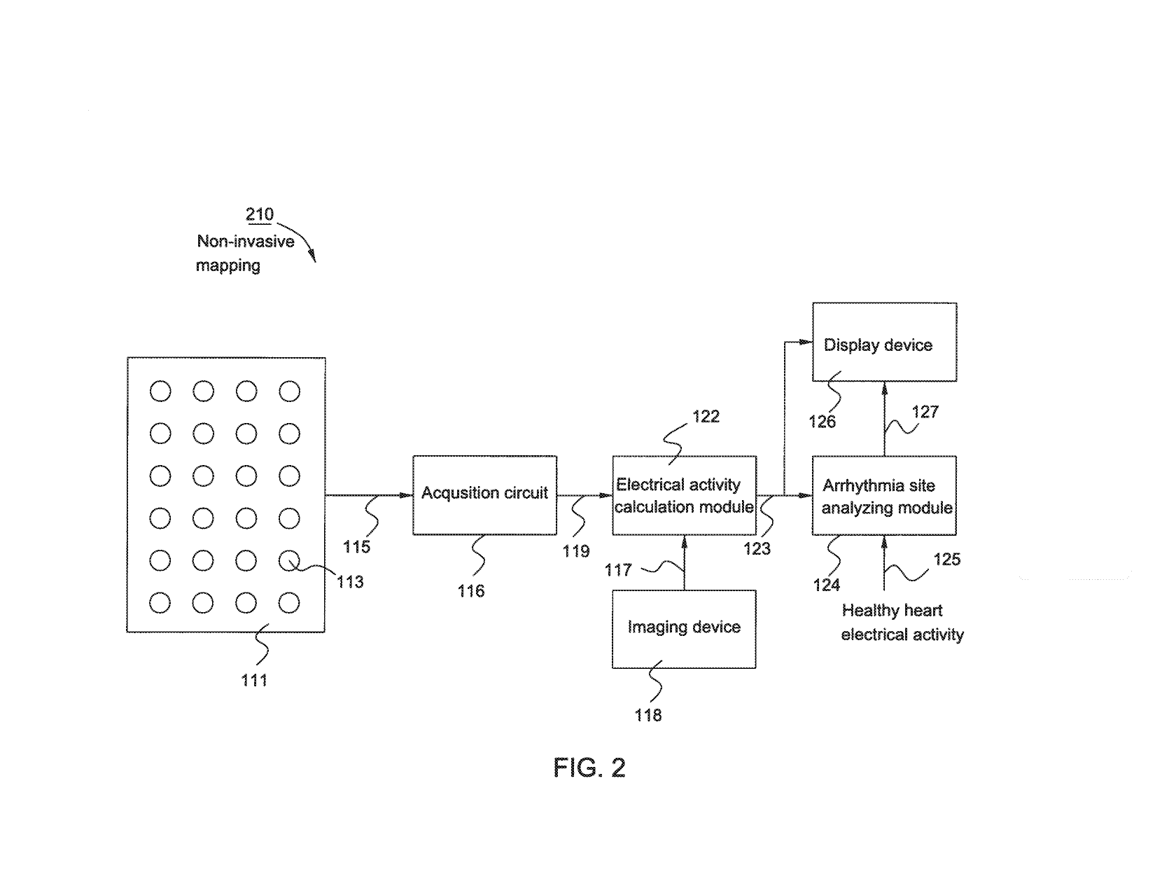

FIG. 2 illustrates a detailed block diagram of a non-invasive mapping system 210 of the cardiac arrhythmia treatment system 100 shown in FIG. 1 in accordance with an exemplary embodiment of the present disclosure. In general, the non-invasive mapping system 210 is configured to localize, determine, or identify the location of the site of origin of cardiac arrhythmias of the human body 200 shown in FIG. 1. More specifically, the non-invasive mapping system 210 includes a sensor assembly 111 which is configured to sense the electrical signals (for example, electrical potentials) 115 at the body surface of the human body 200. In an embodiment, the sensor assembly 111 includes a plurality of electrodes 113 that are placed correspondingly on a plurality of locations of the body surface of the human body 200. More specifically, the plurality of electrodes 113 could be placed uniformly or non-uniformly at the anterior and the posterior of the human body 200. The non-invasive mapping system 210 further includes an acquisition circuit 116 for receiving and conditioning the electrical potentials 115 received from the plurality of electrodes 113. In an embodiment, the acquisition circuit 116 may include a signal amplifier for providing an amplified signal with appropriate amplitude, an analogy-to-digital circuit for digitizing the electrical potential signals, and a filter circuit for removing noise signals. The digitized electrical potential signals 119 are provided to an electrical activity calculation module 122 for calculation of the electrical activity within the heart, and particularly for calculation of the electrical activity throughout the three-dimensional volume of the heart.

In the illustrated embodiment of FIG. 2, the non-invasive mapping device 210 further includes an imaging device 118, which is configured to perform an imaging scan to acquire image data representing three-dimensional images of the torso and heart of the human body 200. The imaging device 118 reconstructs three-dimensional anatomical models of the torso and heart based on the acquired image data. The reconstructed three-dimensional anatomical models of the torso and heart may include a finite element model of the heart and a finite element model of the torso. In the finite element model of the heart, the electrical activity sources within the heart can be equivalently assigned as a single dipole, two dipoles, or multiple dipoles. In addition, the imaging device 118 is further configured to determine the locations of the plurality of electrodes 113 through the image scan. Examples of types of such imaging device 118 that may be used in various embodiments of the present disclosure include ultrasound, computed tomography (CT), magnetic resonance imaging (MRI), Positron emission tomography (PET), and fluoroscopy. In some embodiments, the imaging device 118 can be further configured to perform a temperature monitoring function for optimizing focused energy delivery. The imaging device 118 still can be configured to generate and emit a focused magnetic field at the heart to stimulate electrical activity in the heart. By doing so, the treatment effect of the arrhythmia can be assessed by calculation of stimulation electrical activity within the heart.

In some embodiments, the non-invasive mapping device 210 further includes an electrical activity calculation module 122 that is configured to calculate the electrical activity within the heart by solving an electrocardiogram inverse problem. More specifically, the electrical activity calculation module 122 receives digitized electrical potential signals 119 provided from the acquisition circuit 116 and data signals 117 representing the reconstructed three-dimensional anatomical torso and heart modes of the heart provided from the imaging device 118. In an embodiment, the electrocardiogram inverse problem solving can be expressed by the following equation:

Y=A·X Eqn. (1),

where X is the body surface electrical potentials, which are acquired from the electrodes 113 of the sensor assembly 111, A is the transfer matrix which represents geometrical relationship between the determined locations of the plurality of electrodes placed on the body surface and the various sites throughout the three-dimensional volume of the heart where electrical activities occurs, where the geometrical relationship is the reconstructed three-dimensional torso and heart anatomical models, and Y is the electrical activity throughout the three-dimensional volume of the heart.

In the illustrated embodiments of FIG. 2, the non-invasive mapping system 210 further includes an arrhythmia site analyzing module 124. The arrhythmia site analyzing module 124 is coupled to receive data signals 123 representing the calculated electrical activity provided from the electrical activity calculation module 122. The arrhythmia site analyzing module 124 also receives data signals 125 representing healthy heart electrical activity as a reference. The arrhythmia site analyzing module 124 then compares the calculated electrical activity with the healthy heart electrical activity to determine or identify the site of origin of the arrhythmias. The data signals 125 representing the normal or healthy electrical activity may be pre-obtained and stored in a memory device. By comparisons of the calculated and healthy electrical activity of the heart, the site of origin of the arrhythmia, for example, an abnormal electrical activity excitation site or an abnormal electrical conduction path in the heart can be identified. For purpose of description, the electrical activity calculation module 122 and the arrhythmia analyzing module 124 are illustrated as separate modules. In some embodiments, the two modules 122 and 124 could be integrated as a single module.

As illustrated in FIG. 2, the non-invasive mapping system 210 further includes a display device 126 that is configured to display the calculated electrical activities together with three-dimensional heart images. The display device 126 may display a series of images showing the propagation sequence of the electrical activity of a healthy heart. From the displayed images of the electrical activity of the abnormal heart and healthy heart, the site of origin of the arrhythmia can be easily identified or determined The display device 126 may also display an image of the heart that has been marked to show the site of origin of the arrhythmia based on the analyzing results 127 received from the arrhythmia site analyzing module 124. For example, the abnormal electrical activity excitation site and the abnormal electrical activity conduction path are both displayed on the display device 126. The display device 126 may be any device, such as cathode ray tube (CRT) or liquid crystal display (LCD), that is capable of displaying graphics and images.

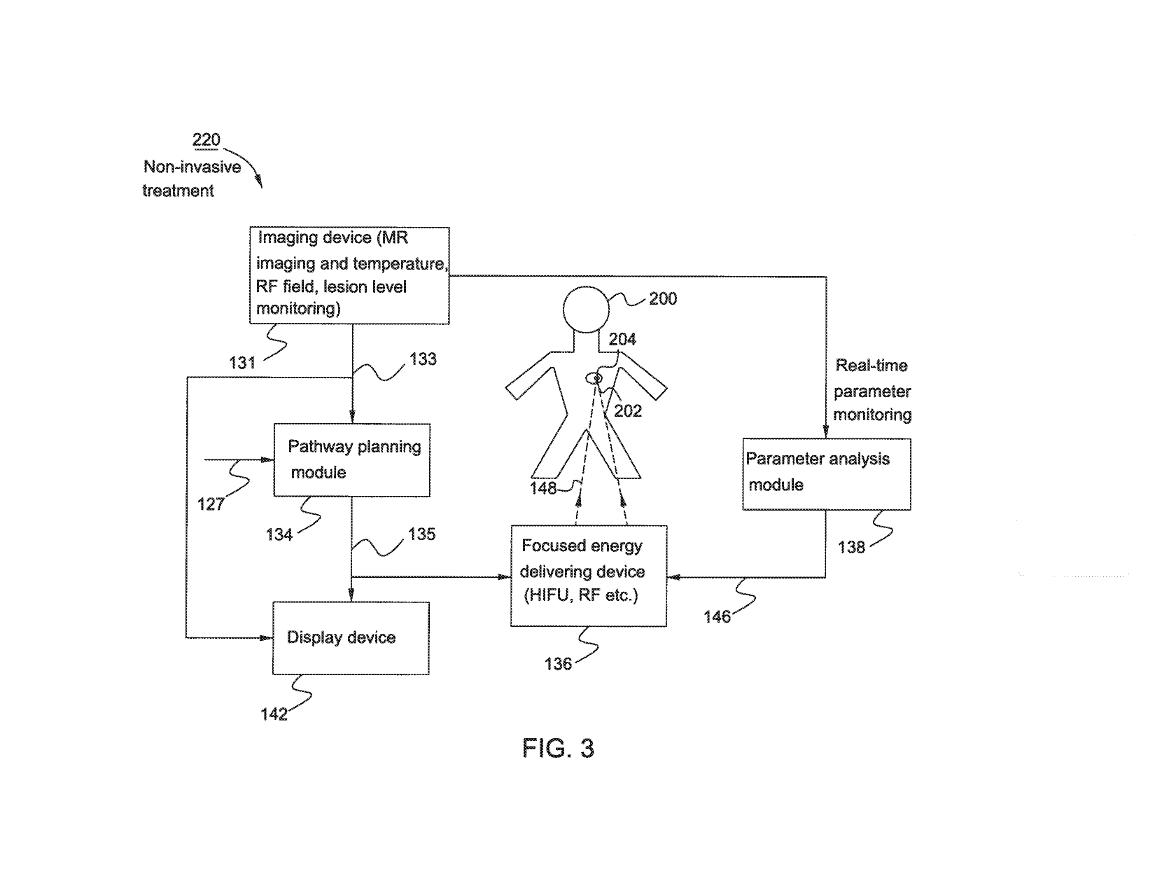

FIG. 3 illustrates a block diagram of a non-invasive treatment system for non-invasive ablation of arrhythmias in accordance with an exemplary embodiment of the present disclosure. The non-invasive treatment system 220 shown in FIG. 3 includes an imaging device 131, a pathway planning module 134, a focused energy delivering device 136, a parameter analysis module 138, and a display device 142. Similar to the imaging device 118 that has been described with reference to FIG. 2, the imaging device 131 shown in FIG. 3 is configured to perform an imaging scan to reconstruct three-dimensional anatomical models of the torso and heart of the human body 200. The imaging device 131 provides data signals 133 representing the three-dimensional anatomical torso and heart models to the pathway planning module 134. The pathway planning module 134 further receives data signals 127 indicating the site of origin of the arrhythmias and provided from the arrhythmia site analyzing module 124 shown in FIG. 2. The pathway planning module 134 then plans or determines a pathway based on the reconstructed three-dimensional torso and heart anatomical models 133 and the identified locations of the site of origin of the arrhythmia 127. In some embodiments, a computational thermal model may be used by the pathway planning module 134 to plan the focused energy pathway. Further in some embodiments, the pathway planning module 134 can be configured to determine an energy focal area for the focused energy. In an embodiment, the planned pathway is a virtual ultrasound energy pathway. In other embodiments, the pathway may be a radio frequency energy pathway, a micro-wave energy pathway, and a laser energy pathway. In some embodiments, the pathway planning module 134 can be further configured to modify the pathway to avoid some critical tissues or structures (for example, blood vessels). The pathway planning module 134 provide data signals 135 to the display device 142 to display images of the planned pathway. In some embodiments, the planned pathway image may be superimposed on the images of the three-dimensional torso and heart models according to the data signals 133 received from the imaging device 131, for facilitating the medical personnel to make appropriate adjustments to the planned pathway according to the images displayed on the display device 142.

In the illustrated embodiment, the data signals 135 representing the planned pathway are further provided to the focused energy delivering device 136. The focused energy delivering device 136 generates and emits focused energy 148 to the site of origin of the arrhythmia 204 of the heart 202 according to the planned pathway 135. The focused energy delivering device 136 can be arranged to be stationary or moveable relative to the human body 200. In an embodiment, the focused energy delivered to the site of origin of the arrhythmia is high-intensity focused ultrasound (HIFU) energy. The HIFU energy penetrates through the intervening tissues and structures and reaches the target sites within the heart. At the target sites, the HIFU energy causes a local temperature increase, resulting in coagulation necrosis of the targeted sites without damaging the surrounding normal tissues or structures. Thus, the arrhythmia can be treated by HIFU energy ablation of the targeted sites. In other embodiments, the focused energy may include radio frequency energy, micro-wave energy, laser energy or any other appropriate energy that could be generated and non-invasively focused at the site of origin of the arrhythmia. In an embodiment, the focused energy delivering device 136 may include a plurality of ultrasound transducer elements (for example, piezoelectric element) for producing ultrasound waves upon electrical signals supplied thereto. In some embodiments, the plurality of ultrasound transducer elements can be arranged to be a phased array manner, in which individual elements can be controlled to interfere adjacent elements to steer the focal point of the focused ultrasound energy.

Further referring to FIG. 3, the dose or amount of focused energy delivered by the focused energy 136 can be varied or adjusted by determining whether the a variety of parameters at the target site receiving the focused energy satisfies predetermined criteria. More specifically, the variety of parameters is particularly monitored to indicate whether a sufficient dose or amount of focused energy is being delivered to the target site to achieve a better treatment of the cardiac arrhythmia. The variety of parameters may include, but are not limited to, temperature, RF current field distribution, lesion level, and tissue elasticity parameters at the target site that focused energy is being delivered.

As illustrated in FIG. 3, the imaging device 131 can be further configured to perform a particular image scan to obtain temperature information of the target site 204 receiving the focused energy. In an embodiment, the imaging device 131 is an MRI device which can use a separate scanning sequence to provide temperature information of the target site 204. In a particular embodiment, a proton resonant frequency (PRF) based MR thermometry method may be used for acquiring the temperature information of the target site 204 receiving the focused energy. In an embodiment, the imaging device or the MRI device 131 provides temperature information 144 to the temperature analyzing module 138. The temperature analyzing module 138 may compare the measured temperature 144 with predefined temperature criteria (for example, a temperature threshold). If the measured temperature 144 is greater than a predefined upper temperature threshold, it is determined that the target site 204 is over heated. In this case, the temperature analyzing module 138 may send control signal 146 to instruct the focused energy delivering device 136 to reduce the intensity of the focused energy being delivered to the target site 204. If the measured temperature 144 is less than a predefined lower temperature threshold, it is determined that the target site 204 is under heated. In this case, the temperature analyzing module 138 may send control signals 146 to instruct the focused energy delivering device 136 to increase the intensity of the focused energy or extend time duration of the focused energy being delivered to the target site 204.

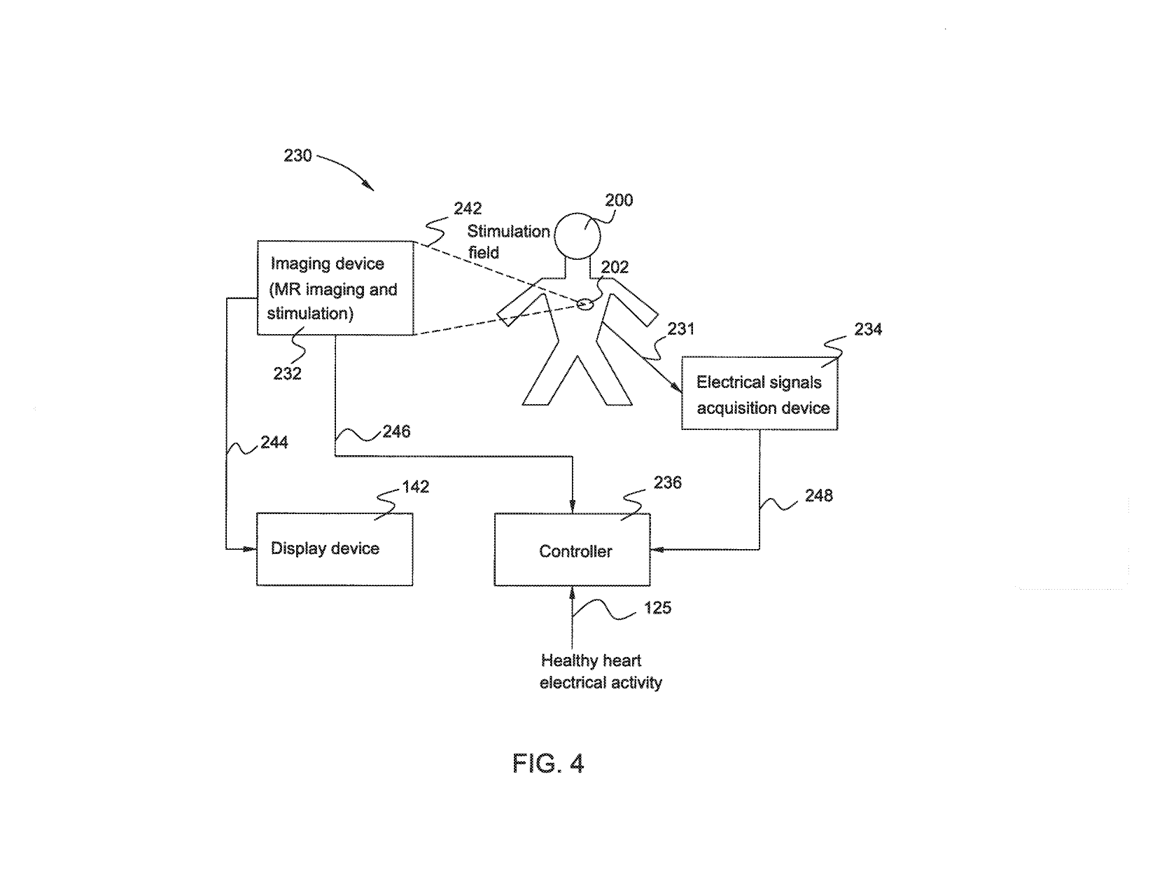

FIG. 4 illustrates a block diagram of a non-invasive assessment system for assessing cardiac arrhythmia in accordance with an exemplary embodiment of the present disclosure. The non-invasive assessment system 230 shown in FIG. 4 may be integrated into the cardiac arrhythmia treatment system 100 as shown in FIG. 1. In other embodiments, the non-invasive assessment system 230 could be implemented as a standalone system. In the illustrated embodiment, the non-invasive assessment system 230 generally includes an imaging device 232, an electrical signal acquisition device 234, and an arrhythmia assessment module 236. The imaging device 232 could be the same as the imaging devices 118 and 131 that have been described with reference to FIG. 2 and FIG. 3, if the non-invasive assessment system 230 is integrated into the cardiac arrhythmia treatment system 100. In an embodiment, the imaging device 232 is an MRI device that is configured to perform an image scan to form three-dimensional anatomical images of the heart 202. The imaging device 232 provides data signals 244 to the display device 142 which may display three-dimensional anatomical images of the heart 202. The imaging device 232 is further configured to generate and emit a stimulation field to stimulate the electrical activity within the heart 202. More specifically, the imaging device or MRI device 232 can be configured to generate and emit a focused magnetic field to a selected location of the heart 202. The heart 202 is stimulated by the focused magnetic field to generate electrical activity which travels through the three-dimensional volume of the heart 202. Similar to the description with reference to FIG. 2 for calculation of electrical activity of an abnormal heart, the stimulated electrical activity can also be calculated by solving an electrocardiogram inverse problem by the arrhythmia assessment module 236 based on electrical signals 231 acquired from an electrical signals acquisition device 234 and reconstructed three-dimensional torso and heart models 246 from the imaging device 232. The arrhythmia assessment module 236 is further configured to determine whether the calculated stimulation electrical activity satisfies predetermined criteria. The arrhythmia assessment module 236 may be configured to receive data signals representing the electrical activity within a healthy heart 202 of the human body 200, and compare the calculated stimulation electrical activity with the healthy electrical activity to determine if the at least one site of origin of the arrhythmia has been properly ablated with focused energy. By this comparison, if the at least one site of origin of the arrhythmia has not been properly ablated, the at least one site of origin of arrhythmia may be further delivered with focused energy to ablate the arrhythmia

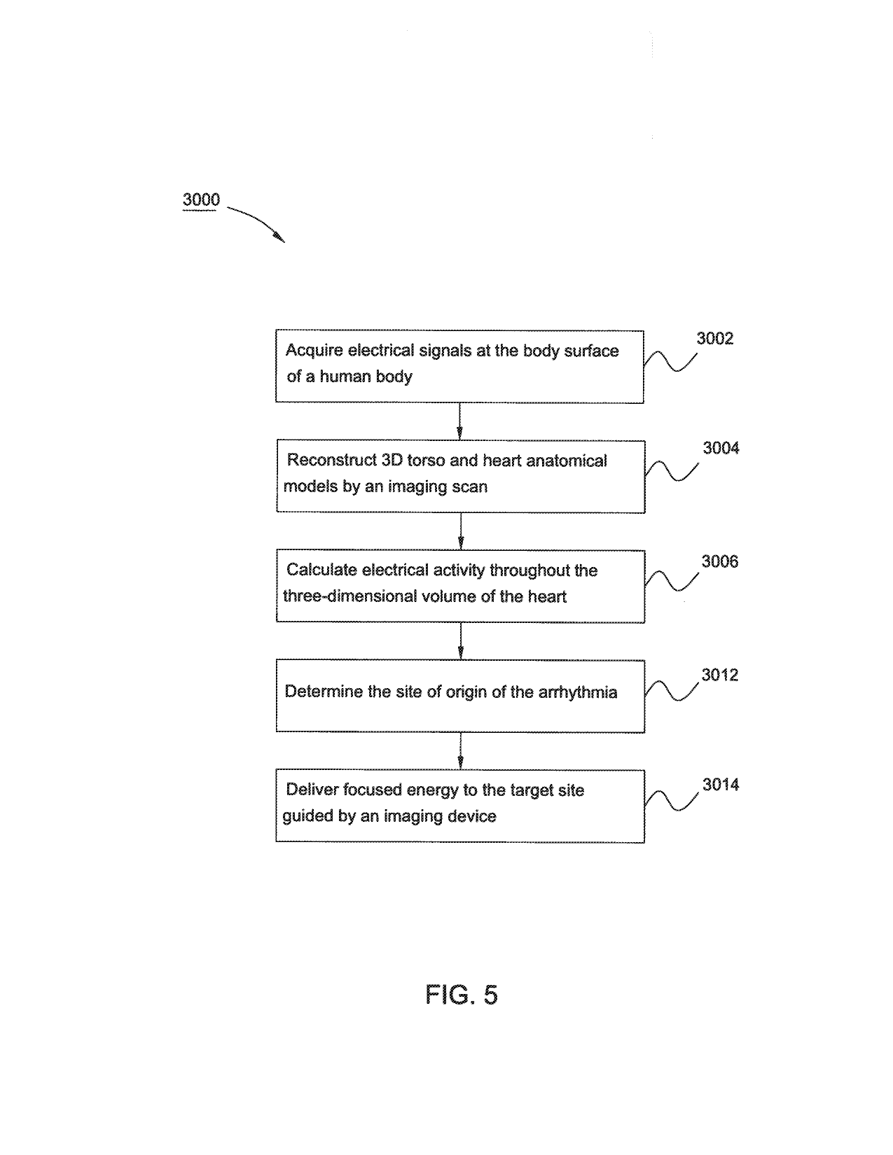

FIG. 5 is a flowchart illustrating a high level method of non-invasive treatment of cardiac arrhythmias in accordance with an embodiment of the present disclosure. As illustrated in FIG. 5, the method 3000 includes a plurality of steps 3002-3014. The detailed implementation of each step 3002-3014 can be tied in some fashion to one or more elements or devices as shown and described with reference to FIGS. 1-4. The method 3000 may be programmed with software instructions stored in a computer-readable medium, which when executed by a processor, performs various steps of the method 3000. The computer-readable medium may include volatile and nonvolatile, removable and non-removable media, implemented in any method or technology. The computer-readable medium includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other non-transitory medium which can be used to store the desired information and which can be accessed by an instruction execution system.

In an implementation, the method 3000 may start to implement at block 3002. At block 3002, electrical signals at the body surface of a human body is acquired. In an implementation, the electrical signals may be electrical potentials generated from the electrical activity within the three-dimensional volume of the heart. The electrical activity within the heart is conducted to the body surface via torso and other body structures. More specifically, the electrical signals may be acquired using an electrical signal acquiring device, such as the sensor assembly 111 having a plurality of electrodes 113 shown in FIG. 2. The electrical signal acquiring device may be combined with an acquisition circuit 116 as shown in FIG. 2 for conditioning the acquired electrical signals. For example, the acquired electrical signals may be amplified, filtered and digitized to provide conditioned electrical signals for the following calculation.

At block 3004, an image scan is performed to reconstruct three-dimensional torso and heart anatomical models of the human body. For example, an MRI device may be used to perform an image scan to acquire imaging data which is used to reconstruct the three-dimensional anatomical models of the torso and heart. Further in some embodiments, the position of the electrodes that are placed on the body surface of the human body can be determined by the image scan. Based on the three-dimensional torso and heart anatomical models, a transfer matrix A representing a geometry relationship between the plurality of electrodes placed on the surface of the human body and origin electrical activity site within the heart can be obtained.

At block 3006, electrical activity throughout the three-dimensional volume of the heart is calculated. In an embodiment, the electrical activity throughout the three-dimensional volume of the heart is calculated by solving an electrocardiogram inverse problem according to the equation (1) as described above with reference to FIG. 2. The electrical activity Y is calculated according to the acquired electrical potentials at the various locations where the plurality of electrodes are placed and the transfer matrix A.

At block 3012, the site of origin of the arrhythmias is determined or identified. More specifically, a determination is made to ascertain the locations of the arrhythmia by comparing the calculated electrical activity throughout the three-dimensional volume of the heart with pre-obtained healthy heart electrical activity. By making the comparisons, the site of origin of the arrhythmia, for example, an abnormal electrical activity excitation site or an abnormal electrical conduction path in the heart, can be identified.

At block 3014, focused energy is non-invasively delivered from outside of the human body to the identified site of origin of the arrhythmias within the heart. In some embodiments, the focused energy is generated and emitted to the target arrhythmia from a high-intensity focused ultrasound (HIFU) device. In some embodiments, the identified locations of the at least one site of origin of cardiac arrhythmia and area surrounding the determined locations on a three-dimensional heart image are visualized on a display device for guiding the HIFU energy delivery to the target arrhythmia sites. The delivered HIFU energy creates a temperature increase at the target arrhythmia site and results in the ablation of the arrhythmias through thermal effects or cavitation effects. In other embodiments, radio frequency energy, microwave energy, and laser energy can be used for the ablation of the arrhythmias. In some embodiments, the focused energy delivery can be guided by an imaging device such as an MRI device or a CT device. Further, an optimized focused energy pathway can be planned or edited by taking into consideration critical structures, such as blood vessels of the human body, such that unintended damage to the critical structures can be avoided.

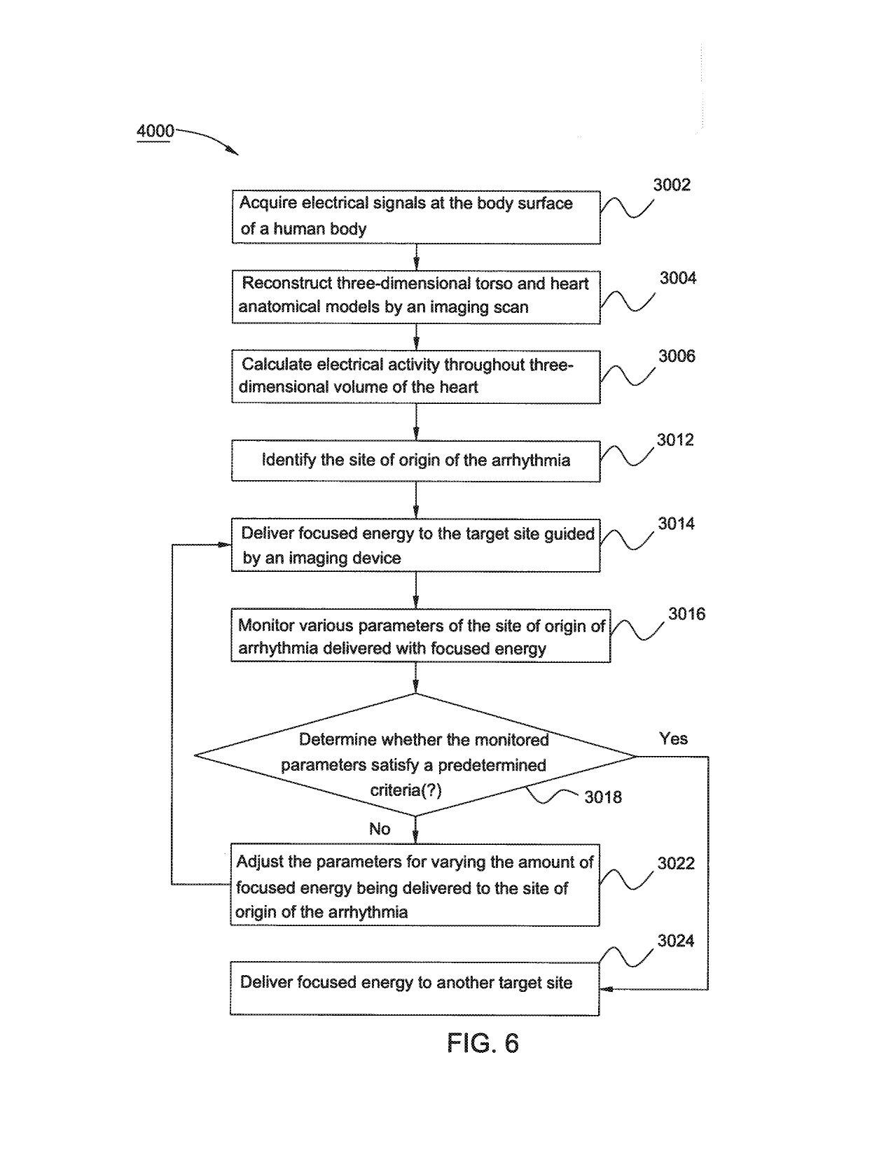

FIG. 6 is a flowchart illustrating a method of non-invasive treatment of cardiac arrhythmia in accordance with another embodiment of the present disclosure. Similar blocks as those shown in FIG. 5 are not described in greater detail in FIG. 6. For example, blocks 3002-3014 for calculating electrical activity within three-dimensional volume of the heart, identifying the arrhythmia sites in the heart, and delivering focused energy for ablation of the arrhythmia sites are omitted. Detailed description will be made to the additional blocks shown in FIG. 6.

In an implementation, following block 3014, the method 4000 further includes a block 3016. At block 3016, parameters in association with a dose or amount of the focused energy being delivered to the target site is monitored. In an embodiment, the temperature at the target site is monitored in real-time or online. That is, the temperature is monitored while the focused energy is being delivered to the target site. Real-time temperature feedback of the target site can help make online parameter adjustments of a focused energy device, such that the arrhythmia of the target site can be more effectively treated. In another embodiment, the temperature information of areas surrounding the target site being treated with focused energy can also be monitored. It is beneficial to obtain the temperature information of the surrounding areas, because unintended damage to these areas can be avoided, for example, by adjusting the focusing area of the focused energy according to the monitored temperature information of the surrounding areas, such that the surrounding area does not become overheated. In an embodiment, as described above with reference to FIG. 3, an imaging device 131 such as an MRI device is used for acquiring the temperature information of the target site. The MRI device 132 can use a separate scanning sequence to provide temperature information of the target site. In alternative embodiments, instead of monitoring the temperature at the target site, other parameters including, but not limited to, RF current field distribution, lesion level, and tissue elasticity at the target site can be non-invasively monitored in real-time to make online adjustments to the focused energy delivering device 140, such that better treatment to the cardiac arrhythmias can be achieved.

At block 3018, whether the monitored or acquired parameter of the target site satisfies predetermined criteria is determined In an embodiment, temperature criteria is set for determining whether a sufficient dose or amount of energy is being delivered. If the monitored or acquired temperature satisfies the predetermined temperature criteria, the procedure goes to block 3024, where focused energy is further delivered to another target site for treatment of the arrhythmia at this another target site. If the monitored or acquired temperature fails to satisfy the predetermined criteria, the procedure goes to block 3022. More specifically, in an embodiment, the predetermined criteria include a predetermined temperature threshold value. In some embodiments, the temperature is desired to be maintained at a specific temperature value. Thus, if the monitored temperature is too high or too low, adjustments should be made to bring the temperature back to the predetermined temperature threshold value. In other embodiments, the temperature criteria may include a time duration threshold that the target tissue should be maintained at a particular temperature.

At block 3022, the parameters of a focused energy device are adjusted to vary the amount of focused energy being delivered to the target site or the site of origin of the arrhythmia. More specifically, in an embodiment, the parameter adjustments may involve increasing the intensity of the HIFU energy when the monitored or acquired temperature at the target site falls below the predetermined temperature threshold value. In another embodiment, the parameter adjustment may involve reducing the focused energy delivery time when the monitored or acquired temperature exceeds the predetermined temperature threshold value.

At block 3024, focused energy is further delivered to another target site until all the arrhythmia sites are treated by focused energy. That is, all the sites that have abnormal electrical activity excitation are terminated or eliminated and the paths that have abnormal electrical activity conduction are terminated or blocked. More specifically, after some sites are treated with optimized parameters for focused energy delivery, the optimized parameters may be directly applied to other sites that are going to receive focused energy for arrhythmia ablation. Although not illustrated in FIG. 6, following or during implementation of block 3024, the method 4000 may further include blocks similar to 3016-3022 shown for online parameter adjustments of the focused energy delivery device through real-time temperature monitoring of the target site that is receiving focused energy.

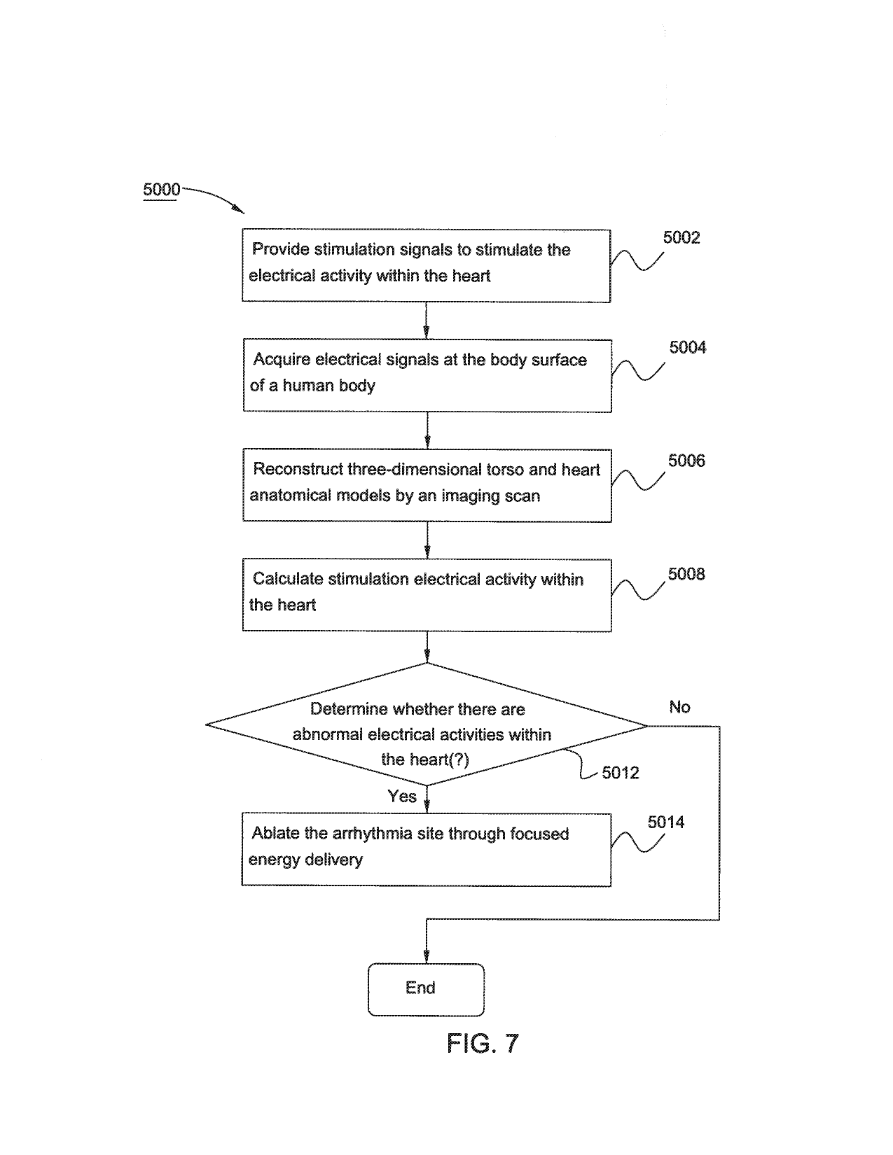

FIG. 7 is a flowchart illustrating a method of non-invasive assessment of cardiac arrhythmia in accordance with an embodiment of the present disclosure. In an embodiment, the method 5000 shown in FIG. 7 may be implemented prior to the cardiac arrhythmia ablation procedure to determine if an arrhythmia ablation needs to be performed. In another embodiment, the method 5000 may be implemented following the arrhythmia ablation procedure to determine whether an arrhythmia ablation is effectively performed.

In the illustrated embodiment, the method 5000 may start from block 5002. At block 5002, stimulation signals are provided to stimulate the heart to generate electrical activity within the heart. In an embodiment, a stimulation device such as an MRI device is used to generate and emit a focused magnetic field at least one location of the heart. The focused magnetic field will induce electrical signals, which propagates throughout the three-dimensional volume within the heart.

At block 5004, electrical signals generated due to the stimulation of the heart are acquired at the surface of the human body. More specifically, the electrical signals such as electrical potentials are acquired by using an electrical signal acquiring device, such as the sensor assembly 111 having a plurality of electrodes 113 shown in FIG. 2. The electrical signal acquiring device may be combined with an acquisition conditioning circuit 116 as shown in FIG. 2 for conditioning the acquired electrical signals. For example, the acquired electrical signals may be amplified, filtered and digitized in order to provide conditioned electrical signals for the following calculation.

At block 5006, an image scan is performed to reconstruct three-dimensional torso and heart anatomical models of the human body. For example, an MRI device may be used to perform an image scan to acquire imaging data which is used to reconstruct the three-dimensional anatomical models of the torso and heart. Further, in some embodiments, the position of the electrodes that are placed on the surface of the human body can be determined by the image scan. Based on the three-dimensional anatomical models of the torso and heart, a transfer matrix A representing a geometry relationship between the plurality of electrodes placed on the surface of the human body and site of electrical activity within the heart.

At block 5008, stimulation of electrical activity throughout the three-dimensional volume of the heart is calculated. In an embodiment, the electrical activity throughout the three-dimensional volume of the heart is calculated by solving an electrocardiogram inverse problem according to equation (1) as described above with reference to FIG. 2. The electrical activity Y is calculated according to the acquired electrical potentials at block 5004 and the transfer matrix X obtained at block 5006.

At block 5012, determination is made to ascertain whether there are abnormal electrical activities throughout the three-dimensional volume of the heart. More specifically, a healthy heart's electrical activity may be pre-obtained by solving an electrocardiogram inverse problem when the heart is at normal or healthy condition. Then, by comparing the calculated stimulation electrical activity of the heart with the pre-obtained healthy heart electrical activity, the locations of the abnormal activities can be determined. In some embodiments, the calculated stimulation electrical activity and the healthy heart electrical activity may be further displayed at a display device for facilitating the determination of the locations of the abnormal electrical activity. If the determination at block 5012 is positive, that is there are abnormal electrical activities within the heart or the prior arrhythmia ablation procedure is not effectively performed, the procedure moves to block 5014. If the determination at block 5012 is negative, that there are no abnormal electrical activities within the heart or the prior arrhythmia ablation procedure was effectively performed, the procedure moves to end.

At block 5014, focused energy is delivered to the locations that are determined to have abnormal electrical activities. In some embodiments, the focused energy is delivered guided by an imaging device such as an MRI device 131. Still in some embodiments, an optimized focused energy delivery pathway may be planned by taking into consideration critical structures, such as blood vessels of the human body, such that unintended damage to the critical structures can be avoided. Further, in some embodiments, the parameters of the focused energy delivery device can be adjusted in real-time by monitoring the temperature of the target site that is being delivered with focused energy and/or the temperature of the areas surrounding the target site.

While the disclosure has been described with reference to exemplary embodiments, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof Therefore, it is intended that the disclosure not be limited to the particular embodiment disclosed as the best mode contemplated for carrying out this disclosure, but that the disclosure will include all embodiments falling within the scope of the appended claims.

What is claimed is:

1. A method, comprising:
acquiring body surface electrical signals at locations on a body surface of a living being from electrodes placed on the locations of the body surface;
reconstructing three-dimensional heart and torso anatomical models of a heart and torso of the living being from an imaging scan;
calculating an electrical activity throughout a three-dimensional volume of the heart of the living being by electrocardiogram inverse problem solving based at least in part on the acquired body surface electrical signals and the reconstructed three-dimensional heart and torso anatomical models;
identifying at least one location of at least one site of origin of a cardiac arrhythmia according to the calculated electrical activity within the heart; and
delivering focused energy to the identified at least one location of the at least one site of origin of the cardiac arrhythmia.

2. The method of claim 1, further comprising:
visualizing the at least one location of the at least one site of origin of the cardiac arrhythmia and an area surrounding the at least one location on a three-dimensional heart image;
planning a pathway for focused energy delivery according to the three-dimensional heart and torso anatomical models and a computational thermal model; and
editing the pathway according to critical structures of the living being.

3. The method of claim 1, further comprising:
monitoring a parameter in association with an amount of the focused energy being delivered at the at least one site of origin of the cardiac arrhythmia by a non-invasive parameter monitoring mechanism;
determining whether the monitored parameter satisfies a predetermined criteria; and
varying the amount of the focused energy delivered to the at least one site of origin of the cardiac arrhythmia according to whether the monitored parameter satisfies the predetermined criteria.

4. The method of claim 3, wherein the monitored parameter in association with the amount of focused energy delivery is selected from a group consisting of temperature, radio frequency field distribution, lesion level, and tissue elasticity.

5. The method of claim 1, further comprising assessing the effectiveness of the focused energy delivery by inducing an electrical stimulus provided by a focused magnetic field from outside of the living being.

6. The method of claim 1, wherein the imaging scan used for reconstructing the three-dimensional heart and torso anatomical models of the living being is selected from a group consisting of a magnetic resonance imaging scan, a computed tomography scan, and an ultrasound scan.

7. The method of claim 1, wherein delivering focused energy comprises generating and emitting high-intensity focused ultrasound energy to the at least one location of the at least one site of origin of the cardiac arrhythmia to form a lesion at the at least one location of the at least one site of origin of the cardiac arrhythmia.

8. The method of claim 1, wherein delivering focused energy comprises generating and emitting radio frequency energy to the at least one location of the at least one site of origin of the cardiac arrhythmia to form a lesion at the at least one location of the at least one site of origin of the cardiac arrhythmia.

9. A system, comprising:
an imaging device configured to perform an image scan for reconstruction of three-dimensional heart and torso models of a living being;
a processor comprising:
a body surface electrical potential acquiring module configured to acquire body surface electrical signals at locations on a body surface of a living being from electrodes placed on the locations of the body surface;
an electrical activity calculation module configured to calculate an electrical activity throughout a three-dimensional volume of the heart by electrocardiogram inverse problem solving based at least in part on the acquired body surface electrical signals and the three-dimensional heart and torso models; and
a cardiac arrhythmia analyzing module configured to identify at least one location of at least one site of origin of a cardiac arrhythmia based at least in part on the calculated electrical activity throughout the three-dimensional volume of the heart; and
a focused energy delivering device configured to deliver focused energy to the at least one location of the at least one site of origin of the cardiac arrhythmia.

10. The system of claim 9, wherein the processor further comprises:
an imaging processing module configured to visualize the at least one location of the at least one site of origin of the cardiac arrhythmia and an area surrounding the at least one location on a three-dimensional heart image; and
a pathway planning module configured to plan a pathway for focused energy delivery according to the three-dimensional heart and torso anatomical models and a computational thermal model, the pathway module being further configured to edit the planned pathway according to critical structures of the living being.

11. The system of claim 9, further comprising:
a non-invasive temperature monitoring device configured to monitor a temperature of the at least one site of origin of the cardiac arrhythmia that is receiving the focused energy and the area surrounding the at least one site of origin of the cardiac arrhythmia; and
a temperature analyzing module configured to determine whether the temperature satisfies a predetermined criteria, the temperature analyzing module being further configured to send control signals to the focused energy delivering device to vary an amount of the focused energy delivered to the at least one site of origin of the cardiac arrhythmia according to whether the temperature satisfies the predetermined criteria.

12. The system of claim 11, wherein the non-invasive temperature monitoring device comprises a magnetic resonance imaging device.

13. The system of claim 9, wherein the processor further comprises an assessing module configured to assess the effectiveness of the focused energy delivery for ablation of the at least one site of origin of the cardiac arrhythmia by inducing an electrical stimulus provided by a focused magnetic field from outside of the living being.

14. The system of claim 9, wherein the focused energy delivering device is configured to generate and emit high-intensity focused ultrasound energy to the at least one location of the at least one site of origin of the cardiac arrhythmia to form a lesion at the at least one location of the at least one site of origin of the cardiac arrhythmia.

15. The system of claim 9, wherein the focused energy delivering device is configured to generate and emit radio frequency energy to the at least one location of the at least one site of origin of the cardiac arrhythmia to form a lesion at the at least one location of the at least one site of origin of the cardiac arrhythmia.

16. The system of claim 9, wherein the imaging device is selected from a group consisting of a magnetic resonance imaging scan, a computed tomography scan, and an ultrasound scan.

17. A non-transitory computer-readable medium comprising instructions stored thereon, which when executed by a processor of a system for non-invasive treatment of cardiac arrhythmia perform a method comprising:
acquiring body surface electrical signals at locations on a body surface of a living being, from electrodes placed on the locations of the body surface;
reconstructing three-dimensional heart and torso anatomical models of a heart and a torso of the living being from an imaging scan;
calculating an electrical activity throughout a three-dimensional volume of the heart by electrocardiogram inverse problem solving based at least in part on the acquired body surface electrical signals and the three-dimensional heart and torso models;
identifying at least one location of at least one site of origin of the cardiac arrhythmia based at least in part on the calculated electrical activity throughout the three-dimensional volume of the heart; and
delivering focused energy to the at least one location of the at least one site of origin of the cardiac arrhythmia.

18. The non-transitory computer-readable medium of claim 17, wherein performing the method further comprises:
visualizing the at least one location of the at least one site of origin of the cardiac arrhythmia on a three-dimensional heart image;
planning a pathway for focused energy delivery according to the three-dimensional heart and torso anatomical models and a computational thermal model; and
editing the planned pathway according to critical structures of the living being.

19. The non-transitory computer-readable medium of claim 17, wherein performing the method further comprises:
monitoring a parameter in association with an amount of focused energy being delivered at the at least one site of origin of the cardiac arrhythmia by a non-invasive parameter monitoring mechanism;
determining whether the parameter satisfies a predetermined criteria; and
varying an amount of the focused energy delivered to the at least one site of origin of the cardiac arrhythmia according to whether the monitored parameter satisfies the predetermined criteria.

20. The non-transitory computer-readable medium of claim 17, wherein performing the method further comprises assessing the effectiveness of the focused energy delivery for ablation of the at least one site of origin of the cardiac arrhythmia by inducing an electrical stimulus provided by a focused magnetic field from outside of the living being.
